(19) United States
(12) Patent Application Publication
(10) Pub. No.: US 2004/0010166 A1
(43) Pub. Date: Jan. 15, 2004

(54) Preparation of hydroxy compounds

(76) Inventors: Kari Aasbo, Skien (NO); Tom Granli, Vear (NO); Harald Breivik, Porsgrunn (NO)

Correspondence Address: WENDEROTH, LIND & PONACK, L.L.P., 2033 K STREET N. W., SUITE 800, WASHINGTON, DC, 20006-1021, US

(21) Appl. No.: 10/275,552

(22) PCT Filed: May 11, 2001
(86) PCT No.: PCT/NO01/00195

(30) Foreign Application Priority Data
May 12, 2000 (NO) 20002497

(51) Int. Cl. C07C 37/50

(52) U.S. Cl. 568/805

(57) ABSTRACT

A new process for preparing a compound of the general formula ArOH (I) in which Ar represents an aryl or heteroaryl group substituted by at least one electron-donating group.

This invention relates to a process for the preparation of certain hydroxy compounds and, in particular, certain aromatic and heteroaromatic hydroxy compounds.

It is well-known that ethers are generally unreactive compounds. Dialkyl ethers can be cleaved under strongly acidic conditions to form an alcohol by the intermediate formation of a dialkyloxonium salt using a strong acid such as hydrobromic or hydroiodic acid at reflux temperature. Similarly, aryl alkyl ethers are cleaved with hydrogen bromide or hydrogen iodide to yield a phenol and an alkyl halide at temperatures of 120-130° C. However, diaryl ethers, such as diphenyl ether, do not react with hydrogen iodide even at 200° C.

Surprisingly, it has now been found that certain ethers containing at least one aryl or heteroaryl group can be converted to the corresponding hydroxy compound under mild conditions.

According to the present invention there is therefore provided a process for the preparation of a compound of the general formula

ArOH (I)

in which Ar represents an aryl or heteroaryl group substituted by at least one electron-donating group, which comprises reacting a compound of the general formula

ArOR (II)

or a precursor thereof, in which Ar is as defined above and R is an optionally substituted alkyl, aryl or aralkyl group, with an acid comprising at least one short chain alkanoic acid.

The term “precursor” refers to compounds which are readily convertible to compounds of formula II by reaction with an acid according to the process of the present invention. The term “precursor” therefore includes, for instance, compounds containing protecting groups which are easily removable by reaction with an acid to give a compound of formula II and compounds in which substituents on adjacent atoms of the aromatic or heteroaromatic ring together with the interjacent atoms form a ring which can be readily broken on reaction with an acid to give a compound of formula II. Thus, the term “precursor” embraces compounds which are capable of forming compounds of formula II in situ on reaction with an acid according to the process of the present invention and which can then be converted into compounds of formula I without isolation of the intermediate compound of formula II.

Throughout this specification, any alkyl group, unless otherwise specified, may be linear or branched and may contain up to 12, preferably up to 6, and especially up to 4, carbon atoms. Preferred alkyl groups are methyl, ethyl, propyl and butyl. When an alkyl moiety forms part of another group, for example the alkyl moiety of an aralkyl group, it is preferred that it contains up to 6, especially up to 4, carbon atoms. Preferred alkyl moieties are methyl and ethyl.

An aryl group may be any aromatic monocylic or polycyclic hydrocarbon group and may contain from 6 to 24, preferably 6 to 18, more preferably 6 to 16, and especially 6 to 14, carbon atoms. Preferred aryl groups include phenyl, naphthyl, anthryl, phenanthryl and pyryl groups, especially a phenyl or naphthyl, and particularly a phenyl, group. When an aryl moiety forms part of another group, for example the aryl moiety of an aralkyl group, it is preferred that it is a phenyl, naphthyl, anthryl, phenanthryl or pyryl, especially a phenyl or naphthyl, and particularly a phenyl, moiety.

An aralkyl group may be any alkyl group substituted by an aryl group. A preferred aralkyl group contains from 7 to 16, especially 7 to 10, carbon atoms, a particularly preferred aralkyl group being a benzyl group.

A heteroaryl group may be any aromatic monocyclic or polycyclic ring system which contains at least one heteroatom. Preferably, a heteroaryl group is a 5- to 18-membered, particularly a 5- to 14-membered, and especially a 5- to 10-membered, aromatic ring system containing at least one heteroatom selected from oxygen, sulphur and nitrogen atoms. Preferred heteroaryl groups include pyridyl, pyrylium, thiopyrylium, pyrrolyl, furyl, thienyl, indolinyl, isoindolinyl, indolizinyl, imidazolyl, pyridonyl, pyronyl, pyrimidinyl, pyrazinyl, oxazolyl, thiazolyl, purinyl, quinolinyl, isoquinolinyl, quinoxalinyl, pyridazinyl, benzofuranyl, benzoxazolyl and acridinyl groups, especially a pyridyl, fuiryl or thienyl, and particularly a pyridyl group.

When any of the foregoing substituents are designated as being optionally substituted, the substituent groups which are optionally present may be any one or more of those customarily employed in the modification of compounds to influence their structure/activity, stability, bioavailability or other property. Specific examples of such optional substituents include, for example, halogen atoms, nitro, cyano, hydroxyl, cycloalkyl, alkyl, haloalkyl, cycloalkyloxy, alkoxy, haloalkoxy, amino, alkylamino, dialkylamino, formyl, alkoxycarbonyl, carboxyl, alkanoyl, alkylthio, alkylsulphinyl, alkylsulphonyl, alkylsulphonato, arylsulphinyl, arylsulphonyl, arylsulphonato, carbamoyl and alkylamido groups. When any of the foregoing substituents represents or contains an alkyl substituent group, this may be linear or branched and may contain up to 12, preferably up to 6, and especially up to 4, carbon atoms. A cycloalkyl group may contain from 3 to 8, preferably from 3 to 6, carbon atoms. An aryl group or moiety may contain from 6 to 10 carbon atoms, phenyl groups being especially preferred. A halogen atom may be a fluorine, chlorine, bromine or iodine atom and any group which contains a halo moiety, such as a haloalkyl group, may thus contain any one or more of these halogen atoms.

Although the group Ar is defined above as representing an aryl or heteroaryl group substituted by at least one electron-donating group, it will be apparent that the group Ar may also be substituted by one or more other substituents at any of the remaining vacant substitution sites. Such additional substituents may be selected from the list of optional substituents given above. Preferred additional substituents include alkyl, hydroxyl and alkoxy groups, especially C₁₋₄ alkyl, hydroxyl and C₁₋₄ alkoxy groups.

Of these, methyl, tert-butyl, hydroxyl and methoxy groups, especially tert-butyl groups, are particularly preferred.

Preferably, Ar represents a C₆₋₁₀ aryl, especially a phenyl or naphthyl, group or a 5- to 10-membered heteroaryl, especially pyridyl, furyl or thienyl, and particularly a pyridyl, group each substituted by at least one electron-donating group.

More preferably, Ar represents a group of the general formula

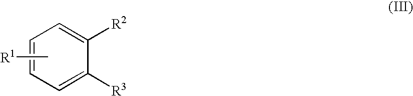

in which R¹ represents an electron-donating group, and R² and R³ independently represent a hydrogen atom or an optionally substituted alkyl, preferably a C₁₋₄ alkyl and especially a tert-butyl, group or R² and R³ together with the interjacent carbon atoms form an optionally substituted aryl, preferably C₆₋₁₀ aryl and especially phenyl, group. It is particularly preferred that Ar represents a phenyl group substituted by at least one electron-donating group.

Preferably, the or each electron-donating group in the compound of formula II is located in the ortho or para position relative to the group —OR. When two electron-donating groups are present, it is preferred that one of these is located in the ortho position relative to the group —OR and the other is located in the other ortho position or the para position relative to the group —OR.

An electron-donating group is any atom or group which enhances the availability of electrons about the aromatic or heteroaromatic ring to which it is attached. Typical electron-donating groups thus include substituents which have an unshared pair of electrons, especially on the atom of the substituent which is adjacent to the ring, which can stabilise intermediates in the reaction by interacting with the n electrons of the ring in an electron-donating resonance effect. Electron-donating groups also include substituents which are capable of polarising the bonding electrons of the ring thus giving rise to an electron-donating polar or polarisability effect.

Preferably, electron-donating groups are selected from the group consisting of halogen, alkyl, aryl, hydroxyl, alkoxy, aralkyloxy, aryloxy, acyloxy, alkylthio, aralkylthio, arylthio, amino, alkylamino, dialkylamino and acylamino groups. In this context, an acyl group is an alkylcarbonyl, aralkylcarbonyl or arylcarbonyl group. It is particularly preferred that the or each electron-donating group is selected from the group consisting of alkyl, hydroxyl., alkoxy, amino, alkylamino and dialkylamino groups, especially C₁₋₄ alkyl, hydroxyl, C₁₋₄ alkoxy, amino, C₁₋₄ alkylamino and di-(C₁₋₄ alkyl)amino groups. More preferably, the electron-donating group or one of the electron-donating groups is a hydroxyl group.

It is preferred that R is an optionally substituted alkyl or aralkyl, especially a C₁₋₄ alkyl or C₇₋₁₀ aralkyl, group. More preferably, R is a methyl or benzyl, especially a methyl, group.

It has been found that the commercial antioxidant BHA (butylated hydroxyanisole) can be converted to the commercial antioxidant TBHQ (tert-butyl-hydroquinone) using the process of the present invention. This is particularly advantageous since TBHQ is more expensive than BHA. Moreover, BHA and TBHQ are currently produced commercially by different synthetic pathways and the commercial advantages of a common synthetic route to both compounds will therefore be readily apparent.

Commercially available BHA is a mixture of 2-tert-butyl-4-methoxyphenol(3-tert-butyl-4-hydroxyanisole) and 3-tert-butyl-4-methoxyphenol(2-tert-butyl-4-hydroxyanisole) and may therefore be represented by the following formula OH

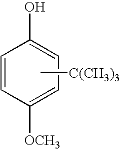

It is therefore preferred that the compound of formula II is 2-tert-butyl-4-methoxyphenol(3-tert-butyl-4-hydroxyanisole), 3-tert-butyl-4-methoxyphenol(2-tert-butyl-4-hydroxyanisole) or a mixture thereof (BHA).

It is also envisaged that the process of the present invention could be useful in the synthesis of other commercial antioxidants which contain aryl or heteroaryl groups substituted by at least one hydroxyl group, particularly antioxidants containing a naphthol or, especially, a phenol group. Specific commercial antioxidants in this respect include BHA, BHT (butylated hydroxytoluene or 2,6-di-tert-butyl-4-methylphenol) and the gallates, particularly the alkyl gallates (alkyl esters of 3,4,5-trihydroxybenzoic acid) and, especially, the ethyl, propyl, octyl and dodecyl gallates. The compound of formula II may therefore have one of the following formulae:—

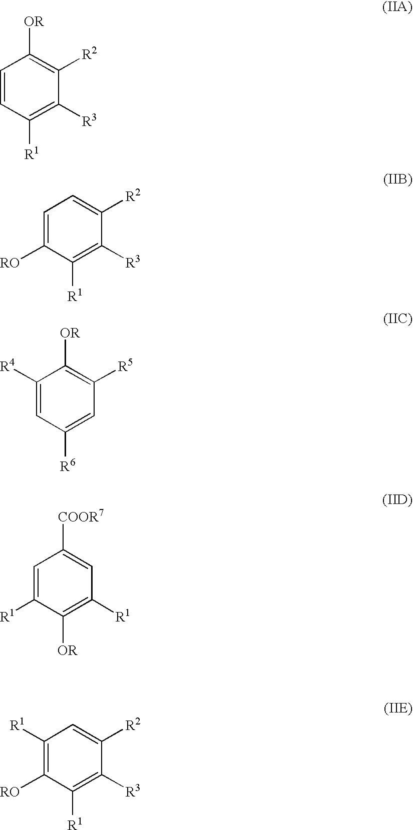

in which R, R¹, R² and R³ are as previously defined, at least one of R⁴, R⁵ and R⁶ is an electron-donating group as previously defined and R⁷ is a hydrogen atom or an alkyl or aralkyl group.

In formulae IIA and IIB, it is preferred that R is an alkyl, especially a methyl, or an aralkyl, especially a benzyl, group and R¹ is a hydroxyl or methoxy group. It is also preferred that one of R² and R³ is an alkyl, especially a tert-butyl, group and that the other of R² and R³ is a hydrogen atom. It is particularly preferred that the compound of formula IIA is BHA or a precursor of BHA which is capable of forming TBHQ when subjected to the process of the present invention. In another preferred embodiment, the compound of formula IIA is capable of forming BHA when subjected to the process of the present invention. Thus, preferably, the compound of formula IIA is a compound in which R is an alkyl or aralkyl, especially a methyl or benzyl, group, R¹ is an alkoxy or aralkoxy, especially a methoxy or benzyloxy, group, one of R² and R³ is a tert-butyl group and the other of R² and R³ is a hydrogen atom and the reaction is interrupted at an intermediate stage and the product of formula I isolated to give BHA.

In formula IIC, it is preferred that R is an alkyl, especially a methyl, or an aralkyl, especially a benzyl, group. Preferably, R⁴, R⁵ and R⁶ each independently represent an alkyl, especially a C₁₋₄ alkyl group. More preferably, R⁴ and R⁵ each represent a tert-butyl group and R⁶ represents a methyl group. It is particularly preferred that the compound of formula IIC is capable of forming BHT when subjected to the process of the present invention.

In formula IID, it is preferred that R is an alkyl, especially a methyl, or an aralkyl, especially a benzyl, group. It is also preferred that each group R¹ is a hydroxyl group. R⁷ is preferably a hydrogen atom or an alkyl group, particularly a C₁₋₁₂ alkyl and, especially, an ethyl, propyl, octyl or dodecyl group, with propyl being particularly preferred. It is particularly preferred that the compound of formula IID is capable of forming a gallate, particularly an alkyl gallate and, especially, propyl gallate when subjected to the process of the present invention.

In formula IIE, it is preferred that R is an alkyl, especially a methyl, or an aralkyl, especially a benzyl, group. It is also preferred that each group R¹ is a hydroxyl group.

The acid utilised in the process of the present invention must comprise at least one short chain alkanoic acid. However, the acid may comprise a short chain alkanoic acid, a mixture of two or more short chain alkanoic acids or a mixture of one or more short chain alkanoic acids and one or more strong acids, such as mineral acids. If a mixture of one or more short chain alkanoic acids and one or more mineral acids is used, it is preferred that the or each mineral acid forms only a minor part of the total acid mixture. Preferably, the total quantity of mineral acid(s) present will not exceed 20%, more preferably 10%, of the total acid mixture.

A short chain alkanoic acid is a carboxylic acid which contains from 1 to 6, preferably 1 to 4, carbon atoms. Most preferably, the short chain alkanoic acid is a C, ₃ alkanoic acid, especially methanoic acid. The alkyl moiety of the short chain alkanoic acid may be substituted by an optional substituent as defined above, especially halogen. However, it is preferred that the alkyl moiety is unsubstituted.

Short chain alkanoic acids are commercially available in various grades of purity. In some circumstances, it may be advantageous to use a substantially pure, that is, 98-100% alkanoic acid. However, it is preferred that the alkanoic acid contains some water. In this respect, 85% methanoic acid is particularly preferred.

The reaction temperature selected will depend on the compound of formula II and the short chain alkanoic acid which are to be used. However, a suitable reaction temperature range is from ambient temperature, for instance, 0° to 30° C., to the reflux temperature of the reaction mixture. In general, the higher the temperature, the faster the reaction proceeds.

Antioxidants of the type described above are utilised in silage aids for the acidic preservation of organic by-products such as fish waste, slaughter waste, poultry waste and food waste in order to protect such products from oxidation. Such silage aids also include at least one acid which is used to treat the raw material to obtain the optimum pH (3.5-4.5) with regard to enzymatic hydrolysis. It is therefore a further advantage of the process of the present invention that an antioxidant such as TBHQ can be produced in an acid solution which can be used without further processing, for instance, as a silage aid in the silage industry. In this respect, it is particularly preferred that the acid used in the process is methanoic acid, especially 85% methanoic acid. Silage aids comprising at least one antioxidant and at least one short chain carboxylic acid form the subject of co-pending International Patent Application No. PCT/NO00/00079 the contents of which are hereby incorporated by reference. However, if the end-product of formula I is to be used in another application, the acid can easily be removed by conventional means.

The invention is further illustrated by the following examples.

### EXAMPLE 1

Preparation of TBHQ

BHA (1.5 g) was dissolved in 85% methanoic acid (100 ml) and the solution was heated with continuous stirring. After refluxing for about 24 hours, analysis by ¹H-NMR spectroscopy revealed that ether cleavage had taken place to produce a solution of TBHQ in 85% methanoic acid.

### EXAMPLE 2

Preparation of TBHQ

BHA was dissolved in 85% methanoic acid to give a concentration of 0.75% by weight BHA and the solution was then stored at room temperature (25° C.). Analysis by HPLC and ¹H-NMR spectroscopy at regular time intervals gave the following results:—

| Elapsed time | % by weight BHA |
|---|---|
| 2 weeks | 0.72 |
| 5 weeks | 0.69 |
| 10 weeks | 0.15 |
| 22 weeks | 0.10 |

Conversion of BHA to TBHQ was confirmed by ¹H-NMR spectroscopy.

### EXAMPLE 3

Preparation of Hydroquinone

Hydroquinone monobenzyl ether (1.5 g) was dissolved in 85% methanoic acid (100 ml) and the solution was heated with continous stirring. After refluxing for about 3 hours, analysis by ¹H-NMR spectroscopy revealed that ether cleavage had taken place to produce a solution of hydroquinone in 85% methanoic acid.

1. A process for the preparation of a compound of the general formula
ArOH (I)
in which Ar represents an aryl or heteroaryl group substituted by at least one electron-donating group, which comprises reacting a compound of the general formula
ArOR (II)
or a precursor thereof, in which Ar is as defined above and R is an optionally substituted alkyl, aryl or aralkyl group, with an acid comprising at least one short chain alkanoic acid.

2. A process according to claim 1 in which Ar represents a C₆₋₁₀ aryl or 5- to 10-membered heteroaryl group substituted by at least one electron-donating group.

3. A process according to claim 1 or claim 2 in which Ar represents a group of the general formula 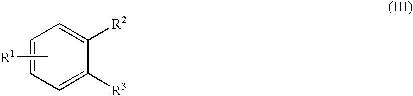
in which R¹ represents an electron-donating group, and R² and R³ independently represent a hydrogen atom or an optionally substituted alkyl group or R² and R³ together with the interjacent carbon atoms form an optionally substituted aryl group.

4. A process according to any one of the preceding claims in which Ar represents a phenyl group substituted by at least one electron-donating group.

5. A process according to any one of the preceding claims in which the or each electron-donating group in the compound of formula II is located in the ortho or para position relative to the group —OR.

6. A process according to any one of the preceding claims in which the or each electron-donating group is selected from the group consisting of alkyl, hydroxyl, alkoxy, amino, alkylamino and dialkylamino groups.

7. A process according to any one of the preceding claims in which the electron-donating group or one of the electron-donating groups is a hydroxyl group.

8. A process according to any one of the preceding claims in which R is an optionally substituted C₁₋₄ alkyl or C₇₋₁₀ aralkyl group.

9. A process according to any one of the preceding claims in which R is a methyl group.

10. A process according to any one of the preceding claims in which the compound of formula II is 2-tert-butyl-4-methoxyphenol(3-tert-butyl-4-hydroxyanisole), 3-tert-butyl-4-methoxyphenol(2-tert-butyl-4-hydroxyanisole) or a mixture thereof (BHA).

11. A process according to any one of the preceding claims in which the acid comprises a short chain alkanoic acid, a mixture of two or more short chain alkanoic acids or a mixture of one or more short chain alkanoic acids and one or more mineral acids.

12. A process according to any one of the preceding claims in which the or each short chain alkanoic acid is a C₁₋₃ alkanoic acid.

13. A process according to any one of the preceding claims in which the acid is methanoic acid.
